(19) United States
(12) Patent Application Publication
(10) Pub. No.: US 2025/0381077 A1
(43) Pub. Date: Dec. 18, 2025

(54) MULTI-FUNCTIONAL SANITARY NAPKIN WITH ADJUSTABLE ABSORBENCY AND SIZE

(71) Applicant: Lauren Rebekah Barna, Palm Beach Gardens, FL (US)

(72) Inventor: Lauren Rebekah Barna, Palm Beach Gardens, FL (US)

(21) Appl. No.: 18/744,643

(22) Filed: Jun. 16, 2024

(51) Int. Cl. A61F 13/472; A61F 13/534; A61F 13/58

(52) U.S. Cl. CPC A61F 13/47227; A61F 13/581; A61F 2013/53445

(57) ABSTRACT

The present invention discloses a multi-functional sanitary napkin designed for customizable menstrual protection. It features a base pad with multiple absorbent layers and a moisture-proof backing for optimal absorption and leak prevention. Removable absorbent sections within the base pad allow users to adjust absorbency by peeling off or detaching sections, each interlocking with adjacent sections to maintain integrity. The napkin includes detachable wings, attached via a perforated seam or snap system, for user preference without compromising structure. Adjustable length and width are achieved through foldable or detachable sections and side panels, secured with fastening mechanisms like Velcro, snaps, or adhesive strips. Re-sealable edges prevent leaks and ensure pad integrity after adjustments. This design prioritizes user comfort, customization, and leak protection, accommodating various needs throughout the menstrual cycle.

### FIELD OF THE INVENTION

The present invention relates to the field of female sanitary napkins. More particularly, the present invention relates to a multi-functional and adjustable sanitary napkin commonly known as maxi pads that can cater to varying body shapes, menstrual flow levels, and personal preferences.

### BACKGROUND OF THE INVENTION

Menstrual pads also known as sanitary pads, sanitary napkins, or female napkins is an absorbent item worn in the underwear when menstruating, bleeding after giving birth, recovering from gynecologic surgery, or in any other situation where it is necessary to absorb a flow of blood from the vagina.

There are several different types of menstrual pads available in the market. For example, panty liners to absorb daily vaginal discharge, light menstrual flow, spotting, slight urinary incontinence, or serve as a backup for tampons or menstrual cups. Ultra-thin pads offer a very compact solution with absorbency comparable to regular or maxi/super pads but with less bulk, providing discreet protection. Regular pads cater to middle-range absorbency needs, suitable for moderate menstrual flow. Maxi/Super pads provide larger absorbency, ideal for the start of the menstrual cycle when menstruation is often heaviest. Overnight pads are longer and offer more protection while the wearer is lying down, making them suitable for overnight use with their enhanced absorbency. Maternity pads are usually slightly longer than maxi/super pads, designed to absorb lochia (post-childbirth bleeding) and can also handle urinary incontinence, offering comprehensive postpartum protection.

Above-mentioned sanitary napkins are available in various sizes, shapes, and absorbency levels to accommodate different flow levels and individual preferences. However, this variety often necessitates the women to purchase multiple types of pads to achieve proper fit and accommodate varying flow levels throughout their menstrual cycle or for urinary incontinence. Typically, women may need to buy 3-5 different types of maxi pads, each designed for specific flow levels and body shapes. This approach not only increases costs but also leads to inconvenience and waste.

Furthermore, many women, in an attempt to find a suitable fit, end up buying pads that do not perfectly match their needs. To address this, some women resort to using cuticle or regular scissors to trim the pads to the desired size and shape. This practice, however, is highly inconvenient and often ruins the integrity of the pad. When a pad is cut, the absorbent material can spill out, and the pad's structure becomes compromised, leading to reduced effectiveness, potential leaks, and discomfort.

Additionally, the disposable nature of these products contributes significantly to environmental waste. The need to purchase and dispose of multiple types of pads exacerbates this issue, highlighting a demand for a more environmentally sustainable solution.

The sanitary napkins available in the market come in fixed sizes and shapes, forcing users to conform to the provided options or alter the pads themselves. This lack of customization results in a less than optimal experience, with users either facing ill-fitting pads or enduring the hassle of manual modifications.

Hence, there is a need for a more versatile solution that can adapt to individual preferences and varying menstrual flow levels without requiring multiple products or damaging alterations.

Therefore, to addresses the above-mentioned shortcomings associated with conventionally available multiple types of sanitary napkins, the present invention discloses a single sanitary napkin or maxi pads with detachable wings that can be easily customized to fit different body shapes and flow levels without compromising its effectiveness and integrity.

### OBJECTIVES OF THE INVENTION

The primary objective of the present invention is to provide a multi-functional sanitary napkin that can be easily adjusted in length, width, and absorbency to fit different body shapes, and accommodate varying menstrual flow levels.

Another objective of the present invention is to provide multi-functional sanitary napkins with removable parts, such as absorbent sections and wings that can be easily detached and re-sealed according to user's needs and preferences.

Another objective of the present invention is to provide a multi-functional sanitary napkin that will closely conforms to the various anatomical shapes of the female urogenital and buttocks region.

Another objective of the present invention is to promote eco-friendly menstrual care by reducing waste generated from the use of multiple single-purpose pads, encouraging the use of a single, adaptable product.

Overall objective of the present invention is to provide a single, adaptable product that enhances comfort, reduces waste, and offers a cost-effective solution for menstrual care.

### SUMMARY OF THE INVENTION

The present invention seeks to eliminate the need for women to purchase multiple types of conventional sanitary napkins by providing multi-functional and multi-sized sanitary napkins.

The said multi-functional sanitary napkin includes removable parts that allows for customizable size, shape, absorbency, and presence of the wings to accommodate various preferences and needs throughout the menstrual cycle.

According to an embodiment, the present invention discloses a multi-functional sanitary napkins having a base pad comprising multiple layers of absorbent material intricately designed to optimize moisture absorption and distribution; a removable absorbent sections strategically placed within the base pads that can be peeled off or detached to adjust absorbency based on user's requirements, wherein each removable sections is interlock with the adjacent section to maintain the structural integrity of the sanitary napkins; and a pair of wings attached to the base pad configured to drape over the side edges of wearer's panties in the crotch portion and be attached to the garment facing side of the wearer's panties in order to provide protection from soiling and so as to keep the napkin properly positioned in the wearer s panties, wherein the said wings are attached through perforated seam to allow users to detach the wing based on user's personal preference. The said detachable wings design caters to individuals who may prefer pads without wings, thereby minimizing potential discomfort or irritation. The said sanitary napkins are characterized by their advanced design, which prioritizes user comfort, customization, and leak protection.

In yet another embodiment, the present invention discloses that the multi-functional sanitary napkins comprises base pad with multiple layers of absorbent material, wherein the each layer of is selected for its absorbency and comfort properties, ensuring optimal performance. The base pad is further reinforced with a moisture-proof backing to provide leakage protection, and dryness throughout use. The inherent flexibility allows the pad to conform seamlessly to the contours of different body shapes, enhancing comfort and reliability.

In yet another embodiment, the present invention discloses that the sanitary napkins comprise various removable absorbent sections with the base pad. The said removable absorbent sections are strategically placed to cater to varying flow needs. Users have the flexibility to adjust the absorbency of the napkin by simply peeling off or detaching these sections. Each section is designed to interlock securely with adjacent sections, maintaining the structural integrity of the pad during use.

In yet another embodiment, the present invention discloses that the sanitary napkins comprises wings that are seamlessly attached via a perforated seam or snap system, allowing users to easily remove them according to the user's need. This feature is particularly beneficial for individuals who prefer pads without wings, reducing potential discomfort or irritation while ensuring a snug fit.

Recognizing the importance of a personalized fit, the invention offers adjustable size and shape features. Length adjustments can be made by folding or detaching sections at designated points, while width adjustments are facilitated through foldable or detachable side panels. Each adjustment point is equipped with a secure fastening mechanism, such as Velcro, snaps, or adhesive, or adhesive strips, ensuring the pad remains securely in place throughout use.

After making adjustments to the pad's size or shape, the remaining edges feature re-sealable elements. These elements prevent leaks and maintain pad integrity during use. Materials such as pressure-sensitive adhesive strips or interlocking fabric layers are utilized to provide reliable sealing capabilities.

These and other embodiment of the present invention will become apparent to those of skill in the art upon review of this specification, including its drawings and claims.

### BRIEF DESCRIPTION OF THE DRAWINGS

FIG. 1 is a top plan view of the multi-functional and multi-size maxi sanitary napkin with removable section attached to the base pad in accordance with an embodiment of the present invention.

FIG. 2 is a top plan view of the multi-functional and multi-size sanitary napkin with some portion of removable section being torn away to accommodate user's requirements in accordance with another embodiment of the present invention.

FIG. 3 is a top plan view of another alternatively preferred sanitary napkins embodiment of the present invention with removable section being torn away to show underlying structure.

FIG. 4 is a top plan view of the multifunctional and multi-size sanitary napkin, wherein the said sanitary napkin is detachable from the base pad through marked dashed line to maintain the size of sanitary napkin as per user's requirements according to an exemplary embodiment of the present invention.

FIG. 5 is top plan of the multifunctional and multi-size sanitary napkin, wherein the said sanitary napkin is detachable from the upper end to fit user's requirements according to another exemplary embodiment of the present invention.

FIG. 6 is a top plan view of the multifunctional and multi-size sanitary napkin, wherein the wherein the said sanitary napkin is detachable from the lower end to fit user's requirements according to another exemplary embodiment of the present invention.

FIG. 7 is a top plan view of the multifunctional and multi-size sanitary napkin, wherein the said sanitary napkin is detachable from the upper and lower end to fit user's requirements according to another exemplary embodiment of the present invention.

FIG. 8 is a cross-section view of the multifunctional and multi-size sanitary napkin, wherein the said sanitary napkins includes a resalable material to maintain the structural integrity of base pad according to another exemplary embodiment of the present invention.

### DETAILED DESCRIPTION OF THE INVENTION

The present invention relates to female sanitary napkins and in particular to sanitary napkins commonly known as maxi pads which is characterized by their unprecedented level of adaptability, comfort, and functionality. Unlike conventional pads, these napkins offer a multitude of features designed to enhance the user experience.

As used herein, the term ‘Sanitary napkin” or “maxi pads” refers to an article which is worn by females adjacent to the pudendal region and which is intended to absorb and contain the various exudates which are discharged from the body (e.g., blood, menses and urine) and which is intended to be discarded after a single use (i.e., it is not intended to be laundered or otherwise restored or reused). Interlabial devices which reside partially within and partially external of the wearer's vestibule are also within the scope of this invention. As used herein, the term “pudendal” refers to the externally visible female genitalia and is limited to the labia majora, the labia minora, the clitoris, and the vestibule.

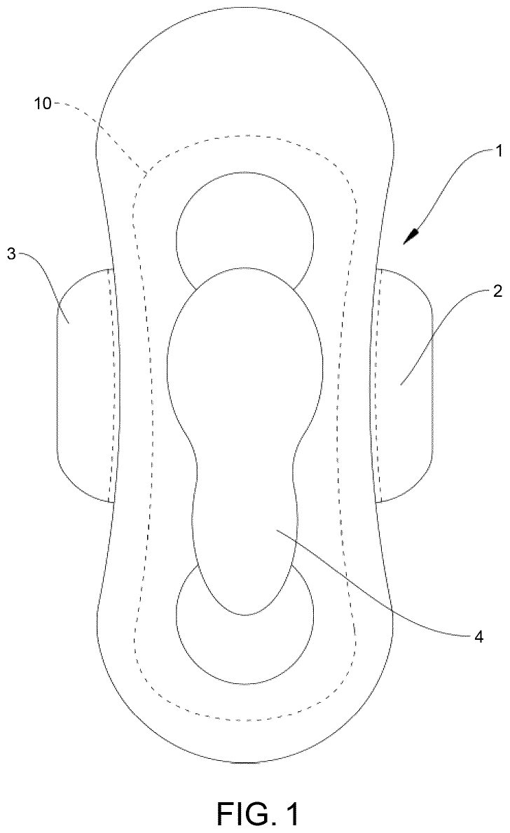

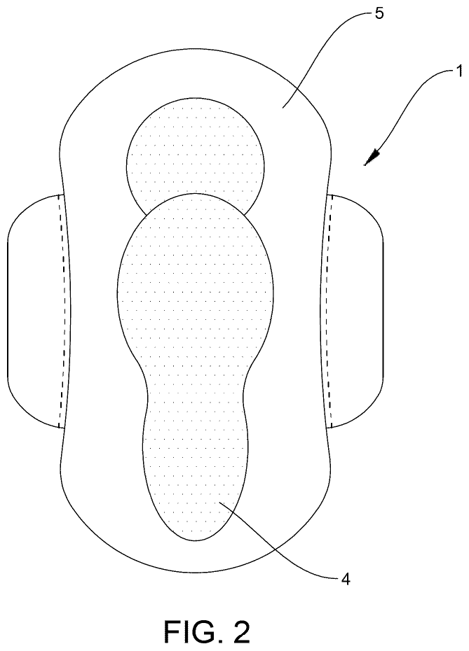

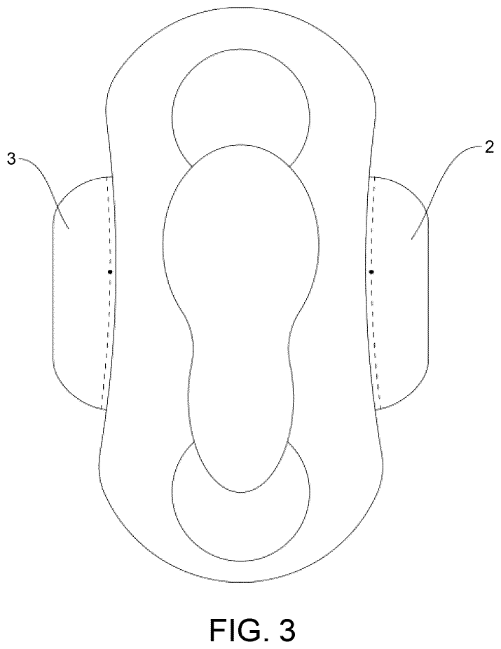

A preferred embodiment of the sanitary napkins of the present invention is shown in FIG. 1-6. As can be seen in FIG. 1-6, preferred sanitary napkins (1) has a body surface (5) which is generally defined by the top sheet and a garment surface which is generally defined by the moisture proof backing (7). The said top sheet comprises a base pad structure (4) made up of multiple layers of absorbent material (6). The said absorbent material may be any means which is generally compressible, comfortable, and non-irritating to the wearer's skin and capable of absorbing and containing body exudates such as menses, blood, and urine. The moisture proof backing (7) is attached below the multiple layer of absorbent material (6) that will come in contact with the wearer's undergarment. The said moisture proof backing (7) may be any material which is flexible and liquid impervious and which prevents the exudates absorbed and contained in the absorbent means from wetting wearer undergarment which contact the sanitary napkin such as panties.

In the preferred embodiment as shown in FIG. 1, the sanitary napkins comprises of at least one removable absorbent section (8) that are interlocked with base pads (4) and other adjacent removable sections (8) to ensure a secure structure when not removed. The said removable absorbent section (8) may be optionally peeled off or detached to accommodate user's requirement based on the contour of female body and their experience on daily menstrual flow. By allowing users to remove sections of the pad, the invention empowers individuals to adjust the absorbency of the napkin to suit their specific needs. Whether experiencing heavy flow or light spotting, users can tailor the absorbency level accordingly, promoting comfort and confidence throughout their menstrual cycle.

Furthermore, the said sanitary napkin has side edges and end edges which together form the periphery of the sanitary napkin. In the preferred embodiment as shown in FIG. 1, wings (2, 3) of the napkins are affixed using either a perforated seam or a snap system. This secure attachment ensures that the wings (2, 3) remain in place during wear, providing reliable support and protection. Users can effortlessly detach the wings (2, 3) from the napkin when desired. Whether through a simple tearing along the perforated seam or unfastening the snap system, this feature allows for quick and hassle-free removal of the wings. Crucially, the removal of the wings does not compromise the overall structure or integrity of the pad. The base pad (4) remains intact and functional even after the wings (2, 3) are detached, ensuring consistent performance and protection. This feature caters specifically to users who prefer pads without wings. By offering the option to remove the wings, the napkin provides enhanced comfort and reduced irritation, accommodating individual preferences and needs.

In yet another embodiment of the present invention, the remaining edges of the sanitary napkins comprises a re-sealable material (9) configured to ensure continued protection and structural integrity throughout use when the removable section of the sanitary napkins is peeled off to accommodate its size and configuration according to user's requirements. The re-sealable material (9) are strategically placed within the sanitary napkins (1) to prevent leaks or seepage, providing users with confidence and peace of mind during wear. This ensures that the pad effectively contains menstrual flow without compromising comfort or hygiene. By incorporating re-sealable elements, the napkin's structure remains intact even after sections are removed or adjustments are made. This feature enhances durability and ensures that the pad remains securely in place throughout use, preventing shifting or displacement. The re-sealable features may utilize materials such as pressure-sensitive adhesive strips or interlocking fabric layers. These materials offer reliable adhesion and sealing properties, maintaining the integrity of the pad and preventing leaks or detachment during wear.

The adjustable size and shape feature of the described sanitary napkin offers users unparalleled flexibility and customization options to ensure a perfect fit and enhanced comfort. Users can easily adjust the length of the pad by either folding or detaching sections at designated points. This allows individuals to tailor the pad to their specific needs, accommodating variations in flow or personal preference. The width of the pad is adjustable through foldable or detachable side panels. This feature allows users to customize the width of the pad to fit their body shape comfortably, providing optimal coverage and leakage protection. Each adjustment point is equipped with a secure fastening mechanism such as Velcro, snaps, or adhesive, or adhesive strips. These mechanisms ensure that the pad maintains its integrity and stays securely in place during wear, even after adjustments have been made.

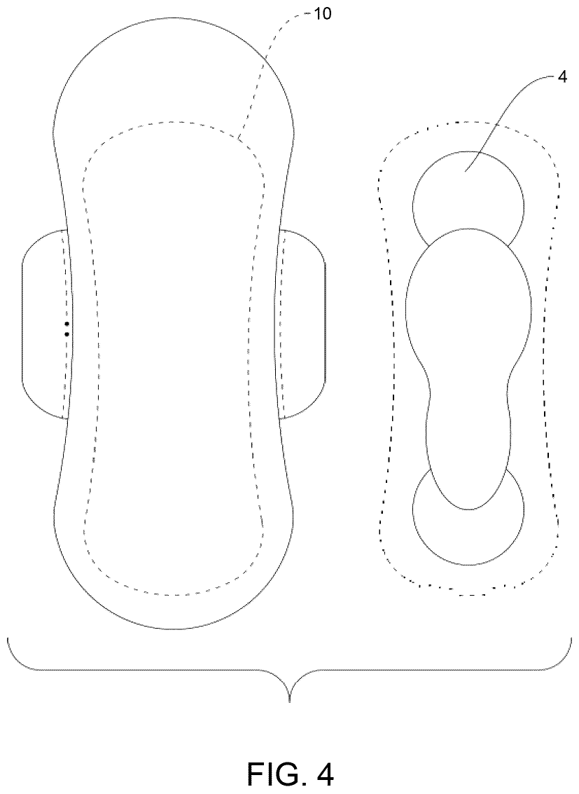

Referring now to FIG. 4 illustrates a top plan view of the multifunctional and multi-size sanitary napkin (1), showing the capability of the sanitary napkin to be adjusted in size according to the user's requirements. The sanitary napkin is detachable along marked dashed lines, allowing for customization of its size. The marked dashed lines (10) indicate predefined sections where the sanitary napkin can be detached from the base pad (4). These lines guide the user to easily remove portions of the napkin, enabling them to adjust the overall length and width of the pad to better suit their personal needs. By following the dashed lines, users can peel off or tear away specific sections of the napkin without compromising the integrity of the remaining parts. This feature allows for personalized sizing and enhanced comfort. The detachment mechanism caters to different menstrual flow levels and body shapes by allowing users to tailor the pad's dimensions.

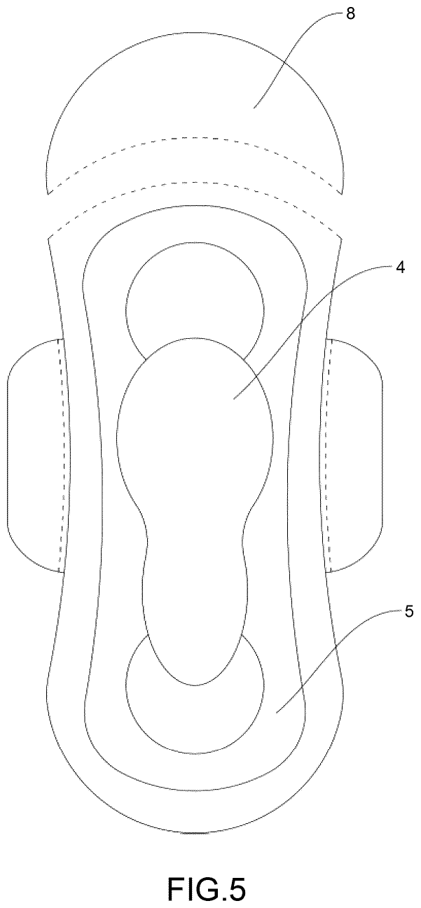

Referring now to FIG. 5, illustrating another top plan view of the multifunctional and multi-size sanitary napkin (1), and demonstrating a different method of adjusting the pad to fit user requirements. Unlike the configuration shown in FIG. 4, this embodiment allows for sections to be detached from the upper part of the sanitary napkin. This provides another level of customization, enabling the user to adjust the absorbent capacity and shape of the pad as needed. The top portion of the sanitary napkin features clearly marked areas where users can detach sections. This flexibility ensures that users can modify the pad's structure to achieve optimal comfort and protection.

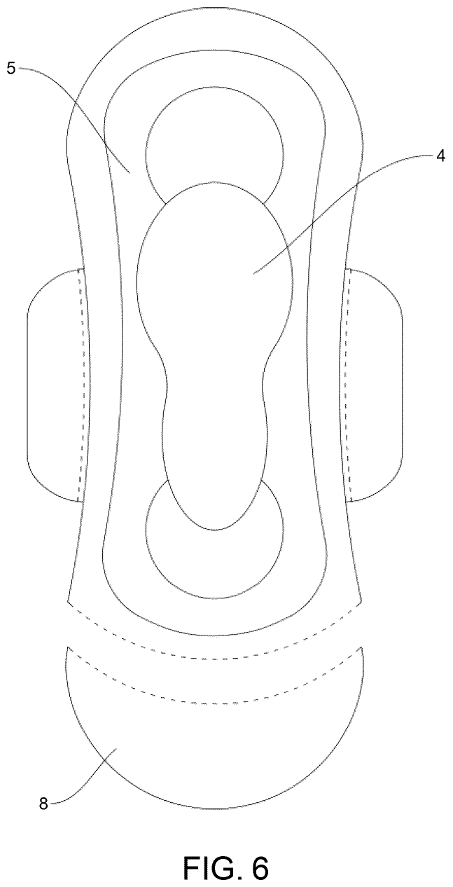

FIG. 6 illustrates a top plan view of another embodiment of the multifunctional and multi-size sanitary napkin (1), where the napkin is detachable from the lower end. Similar to the design in FIG. 5, this embodiment allows users to customize the pad's length by removing the lower section along designated lines. This adaptability ensures that users can achieve a comfortable fit, particularly for varying lengths of wear. By detaching the lower end, users can effectively manage the pad's absorbency and size to suit lighter flow days or specific garment types, all while ensuring no compromise on the pad's performance and comfort.

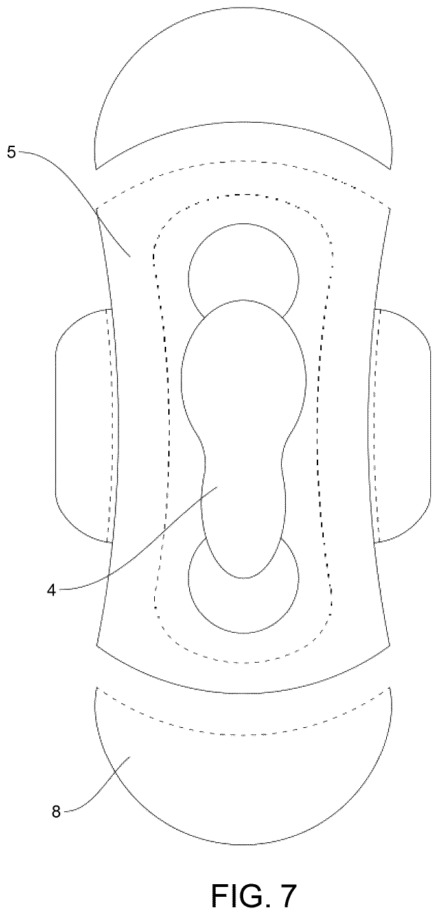

FIG. 7 illustrates a top plan view of the multifunctional and multi-size sanitary napkin (1), offering the most versatile design with detachable sections at both the upper and lower ends. This embodiment provides the highest level of customization, allowing users to adjust both the top and bottom parts of the pad to achieve the desired length and fit. The marked lines guide users in easily detaching sections without affecting the structural integrity of the pad. This dual detachment feature caters to a wide range of needs, from adjusting for different flow intensities to fitting various body contours and clothing styles. It ensures maximum comfort, leakage protection, and confidence throughout the menstrual cycle, demonstrating the innovation's commitment to user-specific adaptability.

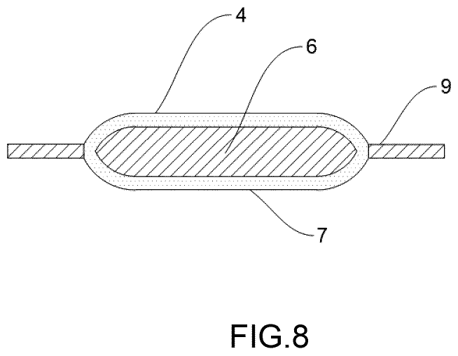

FIG. 8 illustrates a cross-sectional view of the multifunctional and multi-size sanitary napkin (1), highlighting the incorporation of reseal able materials (9) to maintain the structural integrity of the base pad (4). The cross-section reveals layers of absorbent material (6) interspersed with reseal able elements (9). These materials ensure that even after sections of the pad are detached, the remaining structure stays intact and functional. The multiple layers of absorbent material are designed to offer maximum comfort and absorbency. The cross-section demonstrates how these layers work together to absorb menstrual flow effectively while keeping the wearer dry. The bottom layer, identified as the moisture-proof backing (7), ensures that absorbed fluids do not reach the wearer's undergarment, thereby enhancing hygiene and comfort.

In yet another embodiment of the present invention, the base pad structure of the sanitary napkins may be manufactured in a wide variety of sizes and shapes (e.g., rectangular, hourglass, etc.). A preferred shape of the base pad is the dog bone shape shown in FIG. 1. This preferred base pad is 22.0 centimetres long (longitudinal dimension along the longitudinal centerline), 7.0 centimetres wide across its mid portion (lateral dimension along the lateral centerline) and 8.0 centimeters wide across its widest portion (lateral dimension). The base pad is symmetrically configured for ease of manufacture and so that no conscious effort is required by the wearer to properly place the napkin 10 in the direction it should be worn. The mid portion is configured to basically conform to the wearer's thighs and to the thinner crotch portion of the wearer's panties so as to prevent excessive bunching. The size of the base pad may be varied to accommodate wearers ranging in size and also ranging in the expected amount of exudate fluid volume.

I/We claim:

1. A multi-functional (1) sanitary napkins comprising:
a. a body surface (5) defined by a top sheet and a garment surface defined by a moisture-proof backing (7);
b. a base pad (4) made up of multiple layers of absorbent material (6) that is compressible, comfortable, non-irritating to the skin, and capable of absorbing and containing body exudates such as menses, blood, and urine;
c. at least one removable absorbent section (8) interlocked with the base pad (4) and other adjacent removable sections (8), allowing optional peeling or detachment to adjust absorbency according to user requirements; and
d. side edges and end edges forming the periphery of the sanitary napkin, with detachable wings (2,3) affixed via a perforated seam or snap system to drape over the side edges of a wearer's panties and attach to the garment-facing side, providing reliable support and protection while allowing for quick removal without compromising pad integrity.

2. The multi-functional sanitary napkin as claimed in claim 1, wherein the moisture-proof backing is flexible and liquid-impervious, preventing absorbed exudates from wetting the wearer's undergarment.

3. The multi-functional sanitary napkin as claimed in claim 1, wherein the removable absorbent sections are designed to maintain the structural integrity of the sanitary napkin when not removed and are placed strategically to accommodate varying menstrual flow needs.

4. The multi-functional sanitary napkin as claimed in claim 1, wherein the further comprising re-sealable materials on the remaining edges to ensure continued protection and structural integrity after the removal of sections, preventing leaks or seepage and maintaining pad integrity during use.

5. The multi-functional sanitary napkin as claimed in claim 1, wherein the re-sealable materials are selected from the group consisting of pressure-sensitive adhesive strips and interlocking fabric layers.

6. The multi-functional sanitary napkin as claimed in claim 1, wherein the adjustable size and shape features include foldable or detachable sections at designated points to adjust the length and width of the pad, with secure fastening mechanisms such as Velcro, snaps, or adhesive strips ensuring the pad remains securely in place during wear.

7. The multi-functional sanitary napkin as claimed in claim 1, wherein the base pad is manufactured in various sizes and shapes, including rectangular and hourglass, and dog bone.

8. The multi-functional sanitary napkin as claimed in claim 1, wherein the wings, when detached, do not compromise the overall structure or integrity of the pad, ensuring consistent performance and protection.

9. The multi-functional sanitary napkin as claimed in claim 1, wherein the base pad is symmetrically configured to conform to the wearer's thighs and the crotch portion of the wearer's panties, preventing excessive bunching and ensuring ease of placement.

10. The multi-functional sanitary napkin as claimed in claim 1, wherein the detachable wings are characterize to minimize potential discomfort or irritation while providing a snug fit, catering to individual preferences for pads without wings.
